(19) United States
(12) Patent Application Publication
(10) Pub. No.: US 2016/0045561 A1
(43) Pub. Date: Feb. 18, 2016

(54) Antioxidatively Active Composition and the Use Thereof

(71) Applicant: LR HEALTH & BEAUTY SYSTEMS GMBH, Ahlen (DE)

(72) Inventors: Sabine Larsen-Vefring, Berlin (DE); Christian Lendzian, Oelde (DE); Monika Gassen, Troisdorf (DE)

(21) Appl. No.: 14/778,811

(22) PCT Filed: Mar. 19, 2014
(86) PCT No.: PCT/EP2014/055483

§ 371 Date: Sep. 21, 2015

(30) Foreign Application Priority Data
Mar. 21, 2013 (DE) 10 2013 205 049.6

(51) Int. Cl. A61K 36/87; A61K 31/409; A61K 36/886; A61K 36/82

(52) U.S. Cl. CPC A61K 36/87; A61K 36/82; A61K 31/409; A61K 36/886

(57) ABSTRACT

The present invention relates to an antioxidatively active composition including at least one polyphenol rich plant extract; at least one porphyrine, and at least one additional compound from the group of stilbenoids, and the use thereof.

The present invention relates to an antioxidatively active composition according to claim 1 and the use thereof according to claim 17.

Antioxidants have a big physiological importance due to their effect as radical scavenger. They inactivate reactive oxygen species (ROS) in the organism, the presence thereof in excess leading to oxidative stress. Oxidative stress is supposed to be co-responsible for the aging process and is connected to the development of a number of illnesses.

Oxidation and reduction are important biochemical reactions in the human cells. Instable, highly reactive and short lived substances as for instance oxygen radicals arise as intermediate products. Free radicals are understood to be atoms, ions or molecules with one or multiple non-paired electrons in the outer shell. These unpaired electrons have the desire to couple with a further electron and thus have a high reactivity. The term “reactive oxygen species” (ROS) comprises oxygen metabolites and oxygen radicals. On the one hand, ROS have specific physiological functions. They kill germs intracellularly, are active in a regulative manner in the metabolism, control amongst others enzyme reactions and regulate gene expression via redox sensitive transcriptional factors. They, however, can also have a toxic effect, which can cause sub-cellular and cellular damages.

The organism has a complex antioxidative protection system in form of a balanced ratio between synthesis and degradation of reactive oxygen species. If due to disruptions of this balance, an enrichment of ROS occurs, serious functional disturbances can arise. This status is called oxidative stress. Consequences of the oxidative stress are lipidperoxidation, which finally requires that cells have to provide more energy in order to stabilize their membrane potential, protein oxidation and damages of DNA. These three processes are thought to be co-responsible for the aging process and life expectancy.

Endogenic and exogenic factors, which represent an effective protection against free radicals, are present in the organism. They have to establish the balance between required radical formation and radical excess. These compounds are called antioxidants. An antioxidant is a chemical compound which selectively prevents an undesired oxidation of other substances.

Antioxidants have a large physiological importance due to their effect as a radical scavenger. They inactivate within the organism reactive oxygen species (ROS), the presence thereof in excess leads to oxidative stress.

Lifestyle and professional circumstances influence the exogenic load by free radicals. The group of people with increased oxidative stress includes in particular smokers, people with chronical medical intake, competitive athletes, inner city residence, regular strong sunlight/UV exposure, chronical sick people (rheumatic sickness, diabetics) and also airline staff.

When inhaling smoke smokers take up large amounts of free radicals. Due to the ingredients of cigarette smoke free radicals are further created within the organism. Based on studies with vitamin C, it was determined that smokers have an increased requirement of antioxidants, an unfavorable supply with antioxidants and an increased metabolic turnover. The increased radical load of smokers is counteracted by an unfavorable supply with antioxidants.

The intake of medicine in case of sickness is often connected with an increased radical load. The release of oxygen radicals can be either the desired active mechanism (for instance with anti-malaria drugs) or it is an undesired side effect. Cytostatics, Laxatives, Paracetamol or antibiotics as Chloramphenicol can release oxygen radicals by an enzymatic drug substance metabolization. Non-enzymatic drug substance metabolizations are also known, for instance auto-oxidation with the formation of radical intermediate products. This includes amongst others dopa and adrenalin.

Antioxidants are grouped in natural (being in food by nature), nature identical (identical to the natural once in their chemical structure but synthetically obtained) and synthetic antioxidant.

They inhibit unfavorable effects of reactive metabolic products in human, are, however, also added to food since they are able in small concentrations to inhibit the auto-oxidation of easily oxidizable food (for instance fat).

Antioxidants have very different chemical structures. Their common feature is the ability to avoid undesired oxidation and to prevent radical reactions. A reaction partner of oxidation reactions is always oxygen from the air. In the outer electron shell of the oxygen molecule two unpaired electrons with the same spin are present. Thus, it is also a biradical. Thus, oxidation reactions are always radical reactions.

Antioxidatively effective substances are offered in multiple food supplements, for instance as anti-aging preparations and for prevention of sicknesses (for instance cancer), on the market. The contained antioxidative substances are also naturally in the nutrition or are added to food. In particular, food supplements according to the definition of the EU Guideline 2002/46/EG implemented in Germany in the Food Supplement Regulation (NemV) are the group of food which supplement the general nutrition. Their application has to be done in dose form (capsules, tablets, etc.). They contain concentrates of nutrients or other compounds with nutrition specific effect alone or in combination. In the antioxidative prevention, a combination of exogenic antioxidants with a high antioxidative capacity has to be found. Known strong antioxidants are for instance polyphenols, terpenes and phytoestrogens, coenzyme Q10, glutathione, lipic acid, synthetic and natural food additives with antioxidative effect.

The determination of the antioxidative potential can be done using different methods: TAC-Test (Total Antioxidative Capacity), FRAP-Test (Ferric Reducing/Antioxidant Power Essay), TEAC-Test (Trolox Equivalent Antioxidant Capacity Essay), and ORAC-Test (Oxygen Radical Absorbance Capacity Test).

In view of the importance of antioxidants in reducing the risks of chronical diseases, the total antioxidative capacity (TAC) of food and food supplements in adults was investigated 2010 in the USA. Thereby, databases of the US-Department for Agriculture, data of food supplements and food consumption of 4391 US adults in the age above 19 years were evaluated. In order to convert the data for uptake of individual antioxidative compounds to TAC values the measurement of the Vitamin C equivalent (VCE) of 43 antioxidative nutrients was applied beforehand. The daily TAC was on average at 503,3 mg VCE/day, whereby about 75% were taken up by food and 25% by food supplements.

Due to the increasing importance of oxidative stress and its influence on the general wellbeing it was an object of the present invention to develop and provide a composition, which has a high and stable antioxidative capacity.

This object was solved by a composition with the features of claim 1.

Accordingly, the present antioxidative active composition comprises at least one polyphenol-rich plant extract, at least one porphyrine, and at least one additional compound from the group of stilbenoids.

The present antioxidatively active composition has preferably an antioxidative capacity determined in way of an ORAC value of at least 40.000 μmol/l. The ORAC value of the composition is preferably in a range between 40.000 and 80.000 μmol/l, in particular at 40.000 μmol/l.

As mentioned, the ORAC-Test was used at present for determining the antioxidative capacity. The ORAC-Test is based on a fluorometric measurement in which the reactivity of the antioxidants from a sample is measured with respect to peroxyl radicals. The ORAC-Test was at present conducted according to a variant of Chao & Prier et al. (1999). The principle of the method is based on radicals permanently formed by a radical starter. These radicals react with the fluorescent dye fluorescein. The dye is discolored by said radical reaction, i.e. the fluorescence decreases. The decrease of the fluorescence is followed until a complete fluorescence loss. Added antioxidants reduce the degree of the discoloring reaction. The fluorescence decrease is delayed. The degree of the delay serves as a measure for the antioxidative effect of the sample (antioxidants). The measurement is conducted in a fluorescence photometer at 37° C. and 485 nm. The advantage of this method is that hydrophilic as well as lipophilic antioxidants are captured.

In an embodiment of the present composition, the at least one polyphenol rich plant extract is selected from a group containing an extract of green tea, grape juice, red current, cistus, pomegranate, mangosteen, cranberry, cinnamon/cinnamon bark, melissa leafs, roibush, blackberry leafs, raspberry leafs, black tea, linden flowers, marjoran, oregano, peppermint, sage, dried basil, chili powder, clover, cumin, curry, ginger, mustard seed, nut meg, oregano, paprika, parsley, black/red/white pepper, rosemary, thyme, vanilla bean, thyme, black current, rosehip, ginger, pecannut, chocolate, cacao, rice, millet (red, black, etc.).

Polyphenols are comprised of flavonoids in form of flavones, flavanoles, flavonols, anthocyanidines, flavanones, non-flavonoid polyphenols in form of hydroxybenzoic acids, stilbenes and hydroxycinnamon acid. Flavanoids and stilbenes have the same biosynthetic way at the beginning: Condensation reaction of cinnamon acid derivative to styryl-3,5,7-triketoheptane acid. Subsequently, an intramolecular ring closure occurs on the one hand to stilbenoids and on the other hand to flavonoids. According to its developmental stadium of a plant, the involved enzymes regulate either the formation of stilbenes (form resistances against sicknesses) or on the other hand of flavonoids (plant dyes).

A green tea extract is in particular preferably used as a polyphenol rich plant extract. Green tea extract is preferably obtained from a non-fermented green tea of Camellia sinensis. The fresh leafs are dried after harvesting for short time. Oxidative and enzymatic processes are inhibited thereby. The extract is obtained from an aqueous extract and subsequent spray drying. The total polyphenol content is ≧80% and the catechin content ≧60%. The catechin content of green tea is composed of epicatechin (EC), epicatechin gallate (ECG), epigallocatechin (EGC) and epigallocatechin gallate (EGCG). These phenolic compounds cause the extremely high antioxidative capacity of green tea extract in comparison to other plant extracts. Thus, one of the highest concentrations of antioxidative capacity (TAC) amongst herbal teas investigated until now was determined for green tea extract.

In the present composition, the polyphenol rich plant extract, such as the green tea extract, can be used in an amount (mass %) between 0.1 and 5%, preferably 0.1 to 2%, in particular preferably 0.2 to 1%, specifically preferably 0.4%.

In a further embodiment, the at least one porphyrin is selected from a group containing metal containing porphyrins and metal-free porphyrins. As metal containing prophyrins for instance chlorophyll, haemoglobin or vitamin B12 can be used and as metal free porphyrins proto porphyrin, haemato porphyrine or meso porphyrine can be used. Chlorophyll is preferably used, which is richly contained in green vegetables and have a remarkable antioxidative activity in in vitro tests.

The different types of chlorophyll differ in their side groups of the porphyrins. In so far, chlorophylls from different sources are available: cyanobacteria, green algae, brown algae, marine diatom and gold algae, red algae, purple bacteria, green Sulphur and non-sulphur bacteria, green cabbage, stinging nettles, parsley, spinach, broccoli, green beans, green peas, cucumber, kiwis, white cabbage, wheat and barley grass. In case of chlorophyll from wheat grass it also could have been shown that the molecules isolated therefrom can prevent in vitro a DNA damage by oxidation.

The at least one porphyrin as for instance chlorophyll from wheat and/or barley juice powder can be used in the present composition in an amount (mass %) between 0.01 and 1%, preferably between 0.05 and 0.5%, in particular preferably 0.05 and 0.1%, specifically preferably 0.08%.

In a further variant of the present composition, the at least one additional stilbenoid is selected from the group containing stilbenoid alglycons, in particular Piceatannol, Pinosylvin, Pterostilbene, Resveratrol, Stilbenoid-Oligomere, in particular Alpha-viniferin, Ampelopsin A, Ampelopsin E, Diptoindonesin C (isolated from the bark of Shorea pinanga), Diptoindonesin F (isolated from the bark of Shorea gibbosa), Epsilon-viniferin, Flexuosol A, Gnetin H, Hemsleyanol D, Hopeaphenol, trans-diptoindonesin B, Vaticanol B, Stilbenoid-Glycosides, in particular Astringin, Piceid, oligomeric Stilbenoide-Glycosides, in particular Diptoindonesin A. Stilbenes are elements of the defence system of the plant and are synthesized as reaction to stress factors (functional attack, injury by insects, UV radiation).

The use of the stilbenoid resveratrol is in particular preferred. Resveratrol is present in form of four derivatives: (E) and (Z) resveratrol and the glucosides thereof, which are also known as (E) or (Z) piceid. Resveratrol was discovered for the first time in the woods of hellebore. The compound was isolated in 1963 for the first time from the Japanese knot grass (Polygonum Cuspidatum) and identified. Polygonum Cuspidatum has the highest content of resveratrol. It is a plant which originally stems from the traditional Japanese medicine under the name Ko-jo-kon and in the Indian Ayurveda medicine as Darakshasava. Resveratrol can also be obtained from the shell of grapes of the type Vitis Vinifera L., V. Labrusca L. and the leafs. Grape juices contain between 0.1 and 6.5 mg/l resveratrol derivatives (predominantly (E) and (Z) piceids).

Plant synthesize resveratrol via p-Cumaroy coenzyme CoA and Malonyl coenzyme CoA, which are induced often under stress conditions as protective mechanism against fungal, bacteria and virus infections. Resveratrol has besides its antioxidative effect antitumor, anti-rhombic and phytoestrogenic properties. It prevents amongst others the adhesion of thrombocytes. The effectiveness of resveratrol serves also as a possible explanation for the “French Paradoxon”. A phenomenon is described here that Frenchmen despite high alcohol consumption lived longer than other populations (for instance Germans or Americans) and also that the aging phenomena is less prevalent. Furthermore, scientists proved pronounced antimutagenic properties to resveratrol. Thus, it induces phase II drug metabolizing enzymes and inhibits the function of the enzymes cyclooxygenase and hydroperoxidase, which can release dangerous free radicals.

In a further variant of the present composition the at least one additional stilbenoid in form of a plant extract is selected from an extract of Japanese knot grass, grape juice, grape seed, raspberries, mulberries, plums, peanuts. Red wine extracts, extracts from grape wines, cacao beans, bilberries, blueberries and cowberries, cranberries, extracts from jack foods, rhubarb, hops, eucalyptus, different plants from the Lilly family, legumes etc. can be used as further plant extracts. The use of a plant extract from knot wheat and/or from grape juice, in particular white grape juice is thereby preferred.

The plant extract from knot wheat, for instance a 10% resveratrol containing knot wheat extract, can be used in an amount (mass %) between 0.005 and 0.1%, preferably 0.01 and 0.5%, in particular preferably 0.01% in the present composition.

Grape juice, in particular white grape juice, can be used in the present composition in an amount (mass %) between 5 and 50%, preferably 20 and 40%, in particular preferably 30 and 35%.

In a further embodiment, the present composition can contain at least one vitamin selected from the group containing vitamin B1, vitamin B9 (folic acid), vitamin B12 and vitamin E.

Vitamin B12 is also designated as cobalamine. It consists of a ring system of four pyrrole rings. The central atom of this complex ring system is a cobalt atom. Vitamin B12 serves for restauration of the active form of folic acid as methyl folate. Methyl folate is a reaction product during methylation of homocysteine to methionine in the context of DNA synthesis. This function explains the importance of vitamin B12 for rapidly dividing cell tissue, as for instance the cells of the bone marrow.

A mixture of vitamin B1, vitamin B9 (folic acid), vitamin B12 and vitamin E is preferably used, wherein this vitamin mixture can be present in the composition in an amount between 0.01 and 0.2%, preferably 0.05 and 0.1%, in particular preferably 0.08 and 0.1%.

It is furthermore preferred and possible to add to the present composition L-carnitine or at least a salt thereof, preferably L-carnitine tartrate. L-carnitine is as a quaternary ammonium compound by nature an ingredient of the striated muscle and liver. It is a transport substance for acetyl groups mainly between the cytoplasmatic space of the cell and the intramitochondrial space. In this manner, long chain fatty acids can be channeled into the mitochondria. L-carnitine is thus indispensable for fat burning, improves the energy balance, increase the athletic endurance and physical performance.

It is also desirable to add to the present composition trace elements selected from the group containing selenium (Se), chromium (Cr), cobalt (Co), iron (Fe), iodine (I), copper (Cu), manganese (Mn), molybdenum (Mo) and zinc (Zn).

Selenium is in particular preferably added for instance in form of a salt such as sodium selenite. Selenium is for instance contained in glutathione peroxidase and deiodase. Selenium is also designated as cell defensive substance which protects the genetic material against changes. It prevents the lipid peroxidation in membranes of cells and prevents thus the formation of cellular toxic compounds presumably by inhibition due to a radical abortion mechanism. It is a typical antioxidant. Selenium can also be used in form of sodium selenate, sodium hydrogen selenite and/or selenium yeast.

The amount of selenium, in particular sodium selenite used in the present composition is in mass % between 0.005 and 0.015%, preferably 0.005 and 0.01%, in particular preferably 0.007 and 0.009%.

The present composition can further contain choline, preferably as choline bitartrate or choline chloride.

Choline is a biologically important biogenic amine. The transfer agent acetyl choline is biosynthesized from choline and activated acidic acid by the enzyme choline acetyl transferase. As an ingredient of the lecithin choline is a component of all animal and plant cells, in particular biological membranes. The requirement cannot completely be covered in case of increased physical stresses (for instance competitive sport) via food intake and by self-synthesis of the organism.

The amount of used choline in the present composition can be between 0.01 and 0.1%, preferably between 0.01 and 0.5%, in particular preferably between 0.01 and 0.02%.

A quinone derivative, in particular from the group of Ubiquinones, Phylloquinones and/or Pyrrolochinolinequinones PQQ, can also be present in the present composition.

Coenzyme Q10 is preferably used. Coenzyme Q10 is a Ubiquinone. This drug class is constructed on a basic frame of 2,3-dimethoxy-5-methyl-1,4-benzoquinone with an isoprenoid side chain of different lengths of 6-10 dehydroisoprene units. Coenzyme Q10 has 10 isoprene units. Coenzyme Q10 is synthesized in the human organism from phenylalanine. For instance in case of a strongly increased radical load and therewith increased lipid peroxidation the endogenous synthesis of co-enzyme Q10 is not sufficient and should thus be supplemented.

The amount of coenzyme Q10 used in the present composition can be in mass % between 0.05 and 0.15%, preferably between 0.07 and 0.13%, in particular preferably between 0.09 and 0.11%.

The present composition can contain as a further ingredient at least one extract from the plant of stevia. The distribution range of stevia ranges from the Western USA via Central America to Paraguay and Brazil and South America. A plant sweetener is obtained from stevia, the sweetening power thereof is based on steviosides.

The at least one stevia extract can be obtained from one of the following stevial plants: Stevia amambayensis, Stevia ammotropha, Stevia amplexicaulis, Stevia apensis, Stevia aristata, Stevia balansae, Stevia breviaristata, Stevia catharinensis, Stevia commixta, Stevia cuneata, Stevia entreriensis, Stevia estrellensis, Stevia eupatoria, Stevia lemmonii, Stevia leptophylla, Stevia micrantha, Stevia ovata, Stevia parvifolia, Stevia plummerae, Stevia rebaudiana, Stevia rojasii, Stevia sabulonis, Stevia salicifolia, Stevia satureiifolia, Stevia selloi, Stevia serrata, Stevia spathulata, Stevia veronicae, Stevia villaricensis and/or Stevia viscida.

The amount of stevia extract added to the present invention can be (in mass %) between 0.01 and 0.1%, preferably between 0.01 and 0.05%, in particular preferably between 0.01 and 0.03%, specifically preferably 0.2%.

The present invention can contain as a further ingredient at least one sweetener or sugar. The composition contains preferably a monosaccharide, preferably pentoses or hexoses, in particular ribose, arabinose, xylose, glucose, mannose, galactose, fructose, in particular preferably glucose, galactose, fructose.

Monosaccharides as glucose (also known as dextrose or grape sugar) can be immediately resorbed by the human organism without preceding cleavage. Cells of the brain and erythrocytes have to satisfy their energy requirement preferably by glucose in contrast to all other cells of the organism. Glucose serves on the one hand as nutrient and strengthening means and forms the starting product for diabetic sweetener.

The amount of added glucose in the present composition (in mass %) can be between 5 and 15%, in particular preferably 7 and 9%.

It is also possible to add Aloe vera to the present composition. The amount of Aloe vera, that can be used for instance in form of an Aloe vera gel, is in mass % between 10 and 90%, preferably between 20 and 40%, in particular preferably between 30 and 40%.

The use of at least one iron containing compound in the present composition is also of an advantage. For example, iron (III)-pyrophosphate can be added to the composition. Also, iron carbonate, iron citrate, iron ammonium citrate, iron gluconate, iron fumarate, iron sodium diphosphate, iron lactate, iron sulfate, iron diphosphate, iron saccharate, elementary iron (carbonyl+electrolyte+hydrogen-reduced) and/or iron-bisglycinate can be used as iron source. The advantage of taking up iron are manifold for the human. Thus, iron contributes to the reduction of fatigue and tiredness, to a normal cognitive function, to a normal energy metabolism, to normal formation of red blood cells and hemoglobin, to a normal oxygen transport within the body as well as to a normal function of the immune system. Furthermore, iron has a function during cell division.

The amount of iron, in particular of iron(III)-pyrophosphate, in the present composition can be in mass % between 0.01 and 0.1%, preferably between 0.02 und 0.08%, in particular preferably between 0.03 und 0.06%, specifically preferably 0.05%. The iron demand for men is typically 10 mg/day, for women 15 mg/day and can increase for pregnant women and breast feeding woman to 30 mg/day.

Besides the previously mentioned ingredients at least one acidifying agent, preferably citric acid, at least one preservative, in particular potassium sorbate and sodium benzoate, at least one stabilizing agent, preferably Xanthan, natural flavorings as well as at least one further antioxidative means agent, preferably ascorbic acid (vitamin C) can be added in addition to the present composition.

A particular preferred embodiment of the present composition comprises the following ingredients:

| | Amount | Preferred Amount |
|---|---|---|
| Ingredient | [mass %] | [mass %] |
| Aloe vera gel leaf gel | 10-90 | 30-40 |
| sugar/sweetener | 5-10 | 7-9 |
| grape juice | 5-50 | 30-40 |
| green tea extract | 0.1-1 | 0.2-0.5 |
| Xanthan | 0.1-0.5 | 0.2-0.4 |
| L-carnitine compound | 0.1-0.5 | 0.1-0.2 |
| vitamin mixture containing | 0.01-0.1 | 0.05-0.09 |
| vitamin B1, vitamin B12, | | |
| vitamin B9 (folic acid), | | |
| vitamin E | | |
| chlorophyll | 0.01-0.1 | 0.05-0.09 |
| acidifying agent | 0.01-0.1 | 0.05-0.09 |
| preservatives | 0.01-0.05 | 0.02-0.035 |
| flavouring agents, | 0.01-0.05 | 0.01-0.03 |
| coenzym Q10 | 0.05-0.15 | 0.07-0.1 |
| iron compound | 0.01-0.1 | 0.03-0.06 |
| choline compound | 0.01-0.02 | 0.01-0.015 |
| selenium compound | 0.005-0.015 | 0.007-0.01 |
| resveratrol extract | 0.005-0.015 | 0.007-0.012 |

The present composition can be used as means for treatment and/or prophylaxis of stress induced illnesses. The present composition provides an increase of performances, a reduction of tiredness and fatigue, a reduction of oxidative stress, serves the prevention of heart/cardiovascular diseases, arteriosclerosis, diabetes, eye diseases, dementia, cancer and inflammatory diseases as for instance rheumatism, arthrosis, inflammatory bowel diseases.

The present composition serves specifically the reduction of reactive oxygen species (ROS), in particular in human, and thus the prevention of illnesses, caused by ROS and the slowing down of the general aging process in human.

The present composition can also be used as means for increasing the mental performances.

The invention is further explained in more detail by means of examples.

### EXAMPLE 1

A preferred embodiment of the composition according to the invention contains the ingredients as indicated in table 1.

TABLE 1
Ingredients of an embodiment of a composition according to the invention
Charge size 100 kg

| Pos. | Raw Material - Designation | Amount [kg] |
|---|---|---|
| 1 | Aloe vera leaf gel | 36.000 |
| 2 | grape juice concentrate | 32.500 |
| 3 | water | 21.600 |
| 4 | sugar/sweetener | 8.52 |
| 5 | Green tea extract | 0.400 |
| 6 | Xanthan | 0.300 |
| 7 | L-carnitine compound | 0.180 |
| 8 | vitamin mixture of vitamin B1, | 0.0842 |
| | B12, E, folic acid | |
| 9 | chlorophyll | 0.0800 |
| 10 | acidifying agent | 0.096 |
| 11 | preservative | 0.034 |
| 12 | flavouring agent | 0.020 |
| 13 | coenzym Q10 | 0.0904 |
| 14 | iron-compound | 0.0506 |
| 15 | choline compound | 0.0121 |
| 16 | selenium compound | 0.00937 |
| 17 | Resveratrol extract | 0.0100 |

### EXAMPLE 2

The determination of the ORAC value of a composition according to example 2 was carried out in the following manner.

The measuring principle forming the basis of the ORAC measurement is based on the degradation of a fluorescent molecule (at present fluorescein) by free radicals (at present 2,2′-azobis(2-amidinopropane)dihydrochloride, AAPH) and thus the total fluorescence of a sample is reduced. This change serves to illustrate the damage by free radicals. Due to the addition of antioxidants the damage is inhibited, the fluorescence stays intact. The vitamin E derivative trolox serves as a reference, wherefore the result is provided in trolox equivalents.

For sample preparation 1 ml of a sample is at first dissolved in 2.5 ml acetone water mixture (50:50, v/v) and is added up to 10 ml with distilled water (dilution 1:10).

A fluorescein working solution is obtained by diluting a stock solution (c=4,5*10-3 M, 1:200,000). A trolox standard (150 μM, 75 μM, 30 μM, 15 μM) is provided. The test samples are mixed, transferred to 96 microtiterplates and incubated 5 min at 37° C. Subsequently, AAPH is added with instant measurement of the fluorescence at 485 nm (80 times 1.5 min, respectively).

The results obtained by the kinetic measurement are converted into the corresponding trolox equivalents (TE).

### EXAMPLE 3

A user test was conducted for investigating the antioxidative effect of the composition of example 1.

The requirement of an antioxidative supplementation in prevention can be determined using selected markers in urine or in blood/serum/plasma depending on the degree of the oxidative stress. Therefore, the selection of test persons in the user test was done by means of creatinine and isoprostane from urine and/or lipid peroxides from blood.

By means of the present study it was investigated if and to which extend antioxidants in form of the present composition supplied in addition to the normal food with a high ORAC value influence the oxidative stress markers in blood and urine of healthy adult test persons, which have increased oxidative stress markers in blood (lipid peroxide) or urine (isoprostane). It should also be determined, which role the level of the starting values of the oxidative stress markers has on the influence of additional antioxidants and simultaneously the concentration curve of the micro nutrients selenium, coenzyme Q10 and vitamin B12 in the blood should be monitored, which also are added with the present composition.

It is a monocentric, prospective, placebo monitored observational study over three weeks regarding the influence of the daily taken antioxidative micro nutrient-rich drink with an average ORAC value of 40.000 μMol TE/l on the oxidative stress markers: Isoprostane and creatinine in urine, lipid peroxidase in blood as well as the micro nutrients selenium, coenzyme Q10 and vitamin B12 in blood of healthy adults with increased oxidative stress parameters.

The study was conducted in a group of 20 people of both sexes, of which in a randomized manner 15 were assigned to the verumarm (real treatment with actual present active ingredient in contrast to a placebo) and 5 were assigned to the placebo group.

Adult people in the age between 25 and 50 years with a BMI<35 without serious prior medical history with the following oxidative stress parameters were selected: Blood: Lipid peroxides>275 μMol/l and/or urine: 8-epi-prostaglandine>3.5 μg/g creatinine. Test persons with ASA class>2 (for instance insulin required diabetes, malign sicknesses, severe kidney or liver dysfunctions) were excluded from the test.

The following primary measurement values were determined:
Isoprostane test in urine (8-epi-prostaglandine) for determining the load with reactive oxygen compounds (free radicals)
Creatinine in urine as value for the concentration performance of kidney and marker for NO-formation (nitrosative stress)
Lipid peroxide in blood for evaluating an oxidative load (lipid peroxidation)
Selenium, coenzyme Q10, vitamin B12 in blood.

Furthermore, demographic factors (age, sex, date of birth, size in cm and body weight in kg) were recorded.

The test persons took the present composition daily according to the recommended consumption (once 80 ml/day corresponds to 3.200 μMol TE/day).

In the result, the 3-week intake of an antioxidative drink with high ORAC value (40.000 μMol TE/l) reduced the oxidative stress marker in Blood (lipid peroxidase) and urine (isoprostane, creatinine) in healthy adults.

1. An antioxidatively active composition comprising
at least one polyphenol rich plant extract;
at least one porphyrine; and
at least one additional compound from a group of stilbenoids.

2. The composition according to claim 1, wherein the at least one polyphenol rich plant extract is selected from the group consisting of an extract of green tea, grape juice, red currant, cistus, pomegranate, and mangosteen.

3. The composition according to claim 1, wherein the at least one porphyrine is selected from the group consisting of metal containing porphyrines and metal free porphyrines.

4. The composition according to claim 1, wherein the at least one additional stilbenoid is selected from the group consisting of stilbenoid-aglycones, Stilbenoid-Ogligomers, Stilbenoid-Glycosides, and oligomeric Stilbenoide-Glycosides.

5. The composition according to claim 1, wherein the at least one additional stilbenoid used is in a form of a plant extract selected from the group consisting of: an extract of Japanese knotweed, grape juice, grape seed, raspberry, mulberry, plums, and peanuts.

6. The composition according to claim 1, further comprising a vitamin selected from the group consisting of: vitamin B1, vitamin B9 (folic acid), vitamin B12 and vitamin E.

7. The composition according to claim 1, further comprising L-carnatine all at least a salt thereof.

8. The composition according to claim 1, further comprising trace elements selected from the group consisting of: selenium (Se), chromium (Cr), cobalt (Co), iron (Fe), iodine (I), copper (Cu), manganese (Mn), molybdenium (Mo) and zinc (Zn).

9. The composition according to claim 1, further comprising choline.

10. The composition according to claim 1, further comprising at least one quinone derivatives.

11. The composition according to claim 1, further comprising at least one Stevia extract.

12. The composition according to claim 1, further comprising at least one Aloe vera gel.

13. The composition according to claim 1, further comprising at least one monosaccharide.

14. The composition according to claim 1, further comprising at least one iron containing compound.

15. The composition according to claim 1, wherein the composition is effective for treatment or prophylaxis of stress induced diseases.

16. The composition according to claim 1, wherein the composition is effective for increasing mental performance.

17. A method for reducing reactive oxygen species (ROS) in humans comprising:
administering in an effective amount an antioxidatively active composition comprising:
at least one polyphenol rich plant extract;
at least one porphyrine; and
at least one additional compound from a group of stilbenoides.
